(19) United States
(12) Patent Application Publication
(10) Pub. No.: US 2024/0002899 A1
(43) Pub. Date: Jan. 4, 2024

(54) EVALUATION METHOD OF PATHOGEN INACTIVATION EFFECT

(71) Applicant: Institute of Blood Transfusion, CAMS, Sichuan (CN)

(72) Inventors: Zhong Liu, Sichuan (CN); Yundi Yin, Sichuan (CN); Ling Li, Sichuan (CN); Haixia Xu, Sichuan (CN)

(21) Appl. No.: 18/344,501

(22) Filed: Jun. 29, 2023

(30) Foreign Application Priority Data
Jul. 4, 2022 (CN) 202210779876.5

(51) Int. Cl. C12Q 1/18; C12Q 1/70; C12Q 1/06

(52) U.S. Cl. CPC C12Q 1/18; C12Q 1/70; C12Q 1/06; G01N 2333/245; G01N 2333/145

(57) ABSTRACT

The present disclosure provides an evaluation method of a pathogen inactivation effect, including: evaluating an inactivation effect with a maximum value of effective pathogen inactivation (MVEPI) as a standard reference index; where the MVEPI refers to a maximum of a log reduction factor after pathogen solutions of different concentrations before inactivation are treated according to a same inactivation method. The MVEPI can be obtained through the following steps: inactivating a sample containing pathogens with different initial concentrations through a pathogen inactivation method of tested pathogens; detecting changes in the concentration of pathogens with different initial concentrations before and after pathogen inactivation; and analyzing a relationship between the concentration of pathogens before inactivation and the concentration of pathogens after inactivation and the concentration before inactivation by linear fitting and other methods.

### CROSS REFERENCE TO RELATED APPLICATION

This patent application claims the benefit and priority of Chinese Patent Application No. 202210779876.5, filed with the China National Intellectual Property Administration on Jul. 4, 2022, the disclosure of which is incorporated by reference herein in its entirety as part of the present application.

### TECHNICAL FIELD

The present disclosure relates to an evaluation method of a pathogen inactivation effect.

### BACKGROUND

Pathogen inactivation technology is crucial to ensure the biological safety of biological products, food, environment and so on. The effect evaluation and development of the pathogen inactivation technology are inseparable from a reliable detection method for the pathogen inactivation effect.

Pathogen inactivation technology is crucial to ensure the biological safety of biological products, food, environment and so on. The effect evaluation and development of the pathogen inactivation technology are inseparable from a reliable detection method for the pathogen inactivation effect. In 1964, Lowbury et al. proposed the percentage reduction of pathogen counts, that is, a percentage of the number of pathogens reduced by inactivation to the total number of pathogens, to evaluate effects of different skin disinfection methods in the operating room. Accurate counting of pathogen concentrations becomes increasingly difficult as the titer of detected pathogens increases. In 1977, Jean Davies et al. proposed to overcome the difficulty by conducting log conversion of the count, and then proposed the concept of logarithmic reduction factor (log RF) in the effect evaluation of skin disinfection methods in 1978. Specifically, a log value of the total virus count after disinfection is subtracted from a log value of the total virus count before disinfection, and then an arithmetic mean (mean log RF) of the log RF of each sample is calculated. Statistical analysis is conducted to detect a difference between the average log RFs of each reagent, while the pathogen inactivation effect of different techniques is comprehensively evaluated combined with the pathogen count reduction percentage. At present, the pathogen inactivation effect is expressed by log RF in most of the different industries, and still by log RF and/or pathogen count reduction percentage in a few articles.

The two methods currently used, log RF and pathogen count reduction percentage, are indirect evaluation methods. Such methods are difficult to standardize, lack comparability on the evaluation of different inactivation methods, and cannot evaluate a maximum inactivation capacity of different inactivation methods. For example, pathogens with different initial concentrations may result in different log RFs. The pathogens with low concentrations limit the reportable pathogen inactivation effect, while the pathogens with high concentrations may cause pathogens to aggregate and affect pathogen inactivation effects (Forbes, H. A. S., Viral Clearance Strategies for Biopharmaceutical Safety. Pharmaceutical Technology, 2001.25: p. 26-11).

Therefore, there is an urgent need to provide a method for direct evaluation of the pathogen inactivation effect. This method is used to evaluate the maximum inactivation capacity of different technologies and methods, or to compare inactivation capabilities of different inactivation technologies and methods.

### SUMMARY

The existing evaluation methods for pathogen inactivation effect belong to indirect evaluation methods. Such methods give different results using pathogens of different initial concentrations, and cannot reflect a maximum inactivation capacity of inactivation method. As a result, these methods lack standardization and show comparability defects. The present disclosure provides a new evaluation method of a pathogen inactivation effect by comparing inactivation effects with a maximum value of effective pathogen inactivation (MVEPI) as a parameter.

The present disclosure provides an evaluation method of a pathogen inactivation effect, including: evaluating an inactivation effect with an MVEPI as a standard reference index; where the MVEPI refers to a maximum of a log reduction factor after pathogen solutions of different concentrations before inactivation are treated according to a same inactivation method.

Further, the log reduction factor is:

log RF=lg(N/N₀)=lgN−lgN₀; and
log RF represents the log reduction factor, N represents a pathogen concentration before inactivation, and N₀ represents a pathogen concentration after inactivation.

Furthermore, the pathogen concentration before inactivation and the pathogen concentration after inactivation each are detected by counting for in vitro cell culture, or calculated by a PCR method or a chemical method.

Further, the pathogen is selected from the group consisting of a bacterium, a virus, a parasite, a protozoon, and a rickettsia.

Furthermore, the bacterium is selected from the group consisting of Escherichia coli and Staphylococcus aureus, and the virus is vesicular stomatitis virus (VSV).

Further, the pathogen is marked with a label selected from the group consisting of a radioactive label, a fluorescent label, and a chemical label.

Further, the MVEPI is obtained by conducting linear fitting analysis with a pathogen solution concentration before inactivation as an independent variable and a corresponding log reduction factor as a dependent variable; alternatively, conducting linear fitting analysis with the pathogen solution concentration before inactivation as an independent variable and a corresponding pathogen solution concentration after inactivation as a dependent variable.

Furthermore, the MVEPI is obtained by a method including the following steps:
(1) preparing pathogen solutions of different concentrations before inactivation;
(2) conducting pathogen inactivation according to a same inactivation method on each of the pathogen solutions prepared in step (1), and conducting detection to obtain a pathogen concentration in each of the pathogen solutions after inactivation;
(3) calculating a log reduction factor for each of the pathogen solutions:

log RF=lgN−lgN₀; where
log RF represents the log reduction factor, N represents a pathogen concentration before inactivation of the pathogen solution, and No represents a pathogen concentration after inactivation of the pathogen solution;
(4) analyzing a relationship between the lgN and the corresponding log RF thereof, taking a part having a linear function relationship between the lgN and the log RF, and conducting linear fitting with the lgN as an independent variable and the log RF as a dependent variable on the part to obtain a linear fitting function:

D=f(C); where
D represents the log RF, and C represents the lgN; and
(5) allowing D=C in the linear fitting function, and conducting calculation to obtain a value of the C, namely the MVEPI.

Furthermore, the MVEPI is obtained by a method including the following steps:
(1′) preparing pathogen solutions of different concentrations before inactivation;
(2′) conducting pathogen inactivation according to a same inactivation method on each of the pathogen solutions prepared in step (1′), and conducting detection to obtain a pathogen concentration in each of the pathogen solutions after inactivation;
(3′) calculating each of the pathogen solutions of different concentrations before inactivation, and corresponding pathogen concentration logarithm values lgN and lgN₀ in each of the pathogen solutions after inactivation, where N represents the pathogen concentration before inactivation of the pathogen solution, and No represents the corresponding pathogen concentration after inactivation of the pathogen solution;
(4′) analyzing a relationship between the lgN and the lgN₀, taking a part having a linear function relationship between the lgN and the lgN₀, and conducting linear fitting with the lgN as an independent variable and the lgN₀ as a dependent variable on the part to obtain a linear fitting function:

D′=f(C′); where
D′ represents the lgN₀, and C′ represents the lgN₁; and
(5′) allowing D′=0 in the linear fitting function, and conducting calculation to obtain a value of the C′, namely the MVEPI.

Furthermore, the evaluation method further includes conducting error control as follows:
(a′) preparing m parts of the pathogen solution before inactivation that are identical to step (1) as a control group solution according to the method of step (1); and
(b′) subjecting the control group solution obtained in step (a′) and the pathogen solution after inactivation obtained in step (2) to pathogen culture under same conditions for a same time, and comparing a growth rate of pathogens in the control group solution obtained in step (a′) with that of the pathogen solution after inactivation obtained in step (2).

The beneficial effects of the present disclosure are as follows: the evaluation method of a pathogen inactivation effect using MVEPI as a parameter provided by the present disclosure is a method for directly evaluating the pathogen inactivation effect. The method is suitable for evaluating the maximum inactivation capacity of different technologies and methods, or for comparing inactivation capabilities of different inactivation technologies and methods. The method shows wide application range, desirable stability, and accurate and effective evaluation results, and has the value of popularization and application.

In the present disclosure, the term “pathogen concentration” refers to a concentration of surviving pathogens, including pathogen titer, pathogen concentration, or values related to the pathogen concentration detected by various methods. Pathogen count, TCID₅₀, pathogen nucleic acid detection CT value and other values that can be converted to obtain the pathogen concentration all belong to the definition category of “pathogen concentration” in the present disclosure. A method of obtaining the pathogen concentration includes but is not limited to: culturing the pathogen after multiple dilution, calculating the pathogen titer according to a growth state of the pathogen, and then converting to obtain the concentration of the surviving pathogen; adding a chromogenic reagent (bacteria dead/alive dye) to the pathogen, and obtaining the concentration of the surviving pathogen by detecting the chromogenic reagent; conducting direct detection of pathogen-specific survival characterization to obtain the concentration of the surviving pathogen; through flow cytometry or detection of fluorescence intensity of the dye, calculating a percentage of the number of stained bacteria to obtain the concentration of the surviving pathogen. Pathogen concentration is obtained by changing the resonant frequency of a terahertz (THz) metamaterial metasensor coated with a pathogen-free layer as a function of temperature.

In the present disclosure, the concentration gradient design of “pathogen solutions of different concentrations before inactivation” can be routinely adjusted according to the common sense of those skilled in the art, and the function with a linear relationship shall prevail.

Obviously, according to the above-mentioned content of the present disclosure, other various forms of modification, substitution or change can also be made based on the common technical knowledge and conventional means in the art without departing from the above-mentioned basic technical idea of the present disclosure.

The above-mentioned content of the present disclosure will be further described in detail below through the specific implementation in the form of examples. However, they should not be construed as limiting the scope of the above-mentioned subject of the present disclosure to the following examples. All technologies implemented based on the above-mentioned content of the present disclosure fall within the scope of the present disclosure.

### BRIEF DESCRIPTION OF THE DRAWINGS

FIG. 1 shows a change trend of log reduction factor and initial concentration of E. coli in normal saline after RUV treatment.

FIG. 2 shows a change trend of residual titer and initial concentration of VSV in plasma after intermediate-acting RUV treatment.

FIG. 3A shows a change trend of residual pathogen titer with initial concentration of the pathogen.

FIG. 3B shows the results of evaluating an inactivation effect of VSV in plasma by weak RUV using an MLRF method.

FIG. 3C shows the results of evaluating an inactivation effect of VSV in plasma by weak RUV using an MPR method.

FIG. 4A shows results of evaluating inactivation effects of high, medium, and weak RUVs by the MLRF method.

FIG. 4B shows results of evaluating inactivation effects of high, medium, and weak RUVs by the MPR method.

### DETAILED DESCRIPTION

In the present disclosure, raw materials and equipment used are all known commercially available products obtained by purchasing.

### Example 1: MVEPI Value of Inactivation of E. coli by a Riboflavin Inactivation Method and the Effect Evaluation Thereof

### 1. Preparation of Experimental Samples

Fresh overnight-cultured E. coli (K12S lederberg) was suspended after centrifugation and diluted with normal saline to different initial concentrations (1-11 log) for later use.

### 2. Sample Treatment

Inactivation was conducted by referring to a method disclosed in “Yin Y, Li L, Gong L, et al. Effects of riboflavin and ultraviolet light treatment on pathogen reduction and platelets PT Transfusion, 2020, 60(11): 2647-2654.” The saline samples containing different concentrations of E. coli were equally divided into two parts. One part was added with 500 μmol/L riboflavin saline solution at 1:10 as a sample group to be treated, and a final concentration of riboflavin in the sample was 50 μmol/L. The other part was added with the same proportion of normal saline as a control group. The samples of the control group were placed in a 4° C. refrigerator during the treatment of the samples of the experimental group.

200 μL/well of samples from the treatment group containing physiological saline with different concentrations of E. coli were added to a sterile 24-well plate (Thermo Scientific™ Nunc™), and 6 replicate samples were collected for each concentration sample. All covered 24-well plates containing samples were irradiated for 15 min under UV light with a peak at 311 nm.

### 3. Detection After Sample Treatment

After the samples of all test groups were treated, the samples in normal saline control containing different concentrations of pathogens in different experiments (repeatedly taken 6 times) and all samples after treatment were cultured on the corresponding medium after 10× doubling dilution (E. coli were cultured in Brain Heart infusion broth (BHI)), and the growth of pathogens was observed and recorded after 2 d to 4 d. Pathogen titers were calculated by a Reed-Muench method. Pathogen inactivation log reduction factor and pathogen count reduction percentage were calculated using pathogen growth before and after inactivation.

### 4. Experimental Statistics

In each experimental condition, the residual pathogen amount after pathogen inactivation, as well as the calculated log reduction factor after inactivation and the growth amount of the pathogen before inactivation were analyzed for correlation, and there was a correlation when P<0.05. Regression analysis was conducted on relevant data sets, and a significance test was conducted on the regression equation containing constant items by variance analysis. When P<0.05, the difference was considered to be significant, and the regression equation was meaningful.

### 5. Experimental Results

### 5.1 Analysis for Results of the Traditional Evaluation Methods of Pathogen Inactivation Effect

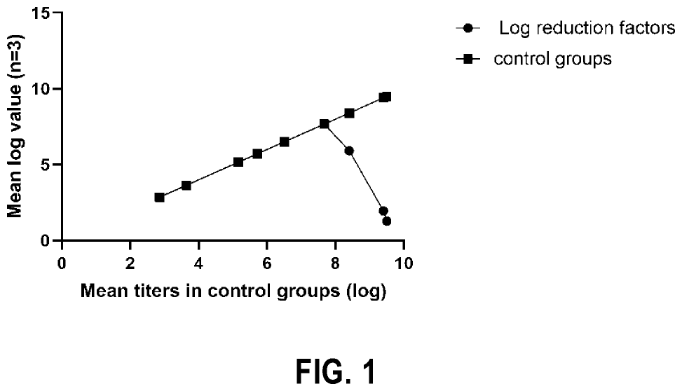

Table 1 and FIG. 1 showed the results of bacterial growth, log reduction factor, and pathogen count reduction percentage of normal saline containing different concentrations of E. coli treated with RUV for 15 min.

TABLE 1
Inactivation effect of riboflavin photochemical method on different
concentrations of E. coli in normal saline (n = 3)

| | | Pathogen | |
|---|---|---|---|
| | Sample pathogen | count | log |
| Initial | concentration | reduction | reduction |
| concentration | after inactivation | percentage | factor |
| 9.50 ± 0.14 | 8.22 ± 0.16 | 96.84 ± 0.37 | 1.50 ± 0.16 |
| 9.40 ± 0.18 | 7.45 ± 0.16 | 99.21 ± 0.00 | 2.10 ± 0.16 |
| 8.40 ± 0.15 | 2.48 ± 0.18 | 100.00 ± 0.00 | 6.15 ± 0.16 |
| 7.67 ± 0.17 | <0.50 | 100.00 ± 0.00 | 7.67 ± 0.17 |
| 6.50 ± 0.20 | <0.50 | 100.00 ± 0.00 | 6.50 ± 0.20 |
| 5.72 ± 0.08 | <0.50 | 100.00 ± 0.00 | 5.72 ± 0.18 |
| 5.16 ± 0.21 | <0.50 | 100.00 ± 0.00 | 5.16 ± 0.22 |
| 3.64 ± 0.17 | <0.50 | 100.00 ± 0.00 | 3.64 ± 0.14 |
| 2.85 ± 0.12 | <0.50 | 100.00 ± 0.00 | 2.85 ± 0.16 |

Notes: when the pathogen residual degree was lower than the limit of detection, the pathogen residual degree was uniformly recorded as 0 in the calculation of inactivation effect in this table.

### 5.2 Proposition of MVEPI Value

It was seen that when the initial concentration of E. coli was less than or equal to 7.67±0.17 log, the log reduction factor of bacterial inactivation increased with the increase of the initial concentration, and no bacterial residue was detected. When the initial concentration of bacteria exceeded 7.67±0.17 log, the amount of residual bacteria increased with the increase of the initial concentration of pathogens, and had a positive linear correlation with the initial concentration, Y=5.115X−40.475 (P=0.026, Y was the residual concentration of E. coli, X was the initial concentration of E. coli). The log reduction factor decreased with the increase of the initial concentration of bacteria, and had a negative linear correlation with the initial concentration, Y=40.475−4.113X (P=0.033, Y was the log reduction factor, and X was the initial concentration of E. coli). Assuming that when the residual amount was approximately to 0, that is, the equation Y=5.115X−40.475=0 (P=0.026, Y was the residual concentration of E. coli, X was the initial concentration of E. coli), or the log reduction factor was equal to the initial concentration, Y=40.475-4.113X=X, the calculated X value represented the MVEPI of E. coli when the residual degree was approximately to 0 under this inactivation condition, both were 7.91.

### Example 2: Evaluation of Inactivation Effect of Riboflavin Inactivation Method on VSV

### 1. Preparation of Experimental Samples

VSV (ATCC® VR-1238 ™) was diluted with normal saline to different concentrations (1-7 log), and then added to plasma at a ratio of 1:10 to obtain plasma containing virus of different concentrations. The plasma was obtained from the Deyang Central Blood Bank and was approved by the local ethics committee.

### 2. Sample Treatment

The sample treatment was divided into three methods: high-acting RUV inactivation VSV treatment, intermediate-acting RUV inactivation VSV treatment, and weak-acting RUV inactivation VSV treatment. The basic processing mode of these methods was the same as that of Example 1, except that the wavelength of light and the dosage were different:

High-acting: 311 nm, irradiation dose 12.98 J/mL; intermediate-acting: 311 nm, irradiation dose 6.49 J/mL; low-acting: 365 nm, irradiation dose 12.56 J/mL. According to the published report “Yin Y, Li L, Gong L, et al. Effects of riboflavin and ultraviolet light treatment on pathogen reduction and platelets [J]. Transfusion, 2020, 60 (11): 2647-2654.”, inactivation effects of inactivation method from high to low was high-acting>intermediate-acting>low-acting.

### 3. Detection After Sample Treatment

The process was the same as that in Example 1

### 4. Experimental Statistics

The process was the same as that in Example 1

### 5. Experimental Results

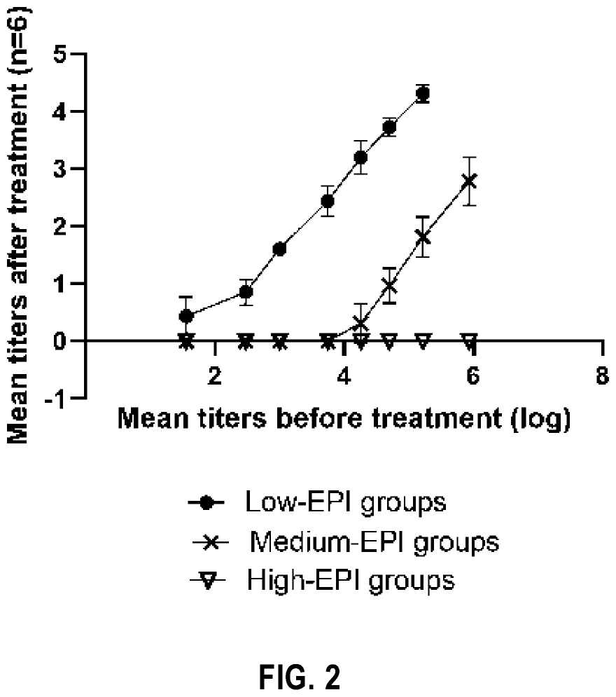

When VSV in plasma was inactivated with intermediate-acting RUV, when the VSV concentration was ≤3.75±0.22, the log reduction factor increased with the increase of the initial concentration. When the VSV concentration≥4.26±0.28, the residual VSV increased with the initial concentration, Y (residual concentration)=1.483X(initial concentration)−5.985 (P=0.001). The log reduction factor decreased with the increase of the initial concentration, and had a negative linear correlation with the initial concentration, Y (log reduction factor)=5.985−0.483X (initial concentration) (P=0.007). FIG. 2 showed a change trend of residual titer and initial concentration of VSV in plasma after acting RUV treatment; the calculated MVEPI values were all 4.04.

Inactivation of VSV in plasma using a weak-acting RUV also demonstrated the applicability of MVEPI. When the VSV concentration was lower than 1.56±0.13, the log reduction factor after inactivation increased with the initial concentration. When the concentration of VSV was ≥2.48±0.25 log, the residual amount of VSV after inactivation increased with the increase of the initial concentration, Y (residual amount)=1.264X (initial concentration)−2.241 (P<0.001). After inactivation, the log reduction factor decreased with the increase of the initial concentration, Y (log reduction factor)=2.241−0.264X (initial concentration) (P<0.001). The MVEPI calculated using both the residual degree and the log reduction factor was 1.77.

When the high-acting RUV inactivated VSV in plasma, the maximum limit of detection was exceeded, that is, the MVEPI exceeded 5.99, and an accurate value could not be obtained.

From the comparison of above results, according to the value of MVEPI: the high-acting MVEPI of greater than 5.99, the intermediate-acting MVEPI of 4.04, and the low-acting MVEPI of 1.77, it was determined that inactivation effect was high-acting>medium-acting>low-acting. These results were consistent with the actual situation reported in the literature, proving that the evaluation method of a pathogen inactivation effect by the MVEPI in the present disclosure was accurate and effective, and showed desirable sensitivity and stability.

### Comparative Example 1: Evaluation Results of MLRF Method and MPR Method

E. coli was inactivated with reference to the method of Example 1. The two most commonly used evaluation methods for inactivation effect were adopted: (1) mean logarithmic reduction factor method (MLRF, expressing inactivation effect with log RF); (2) mean percentage reduction method (MPR, expressing inactivation effect with pathogen count reduction percentage). The accuracy and stability of the above two evaluation methods were verified under different initial concentrations.

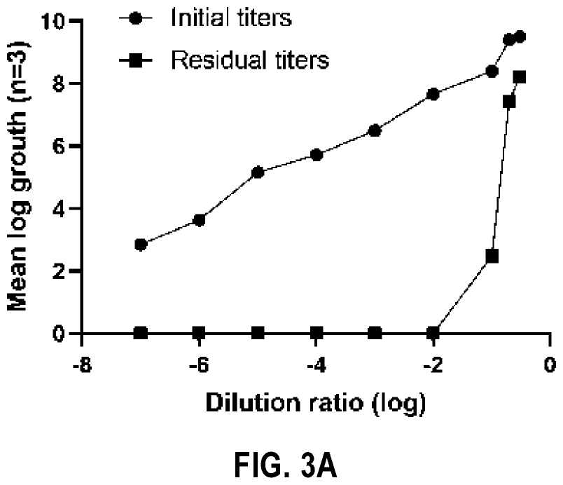

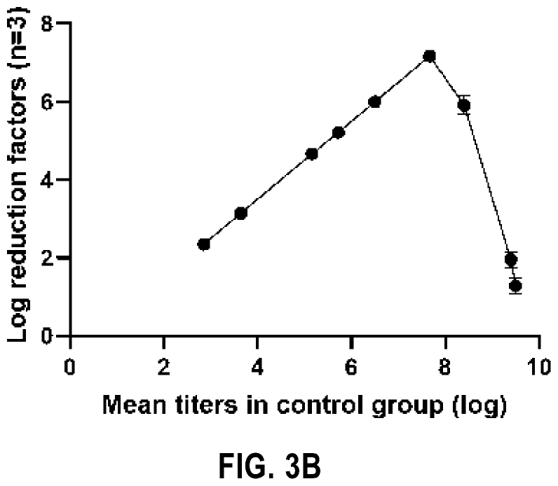

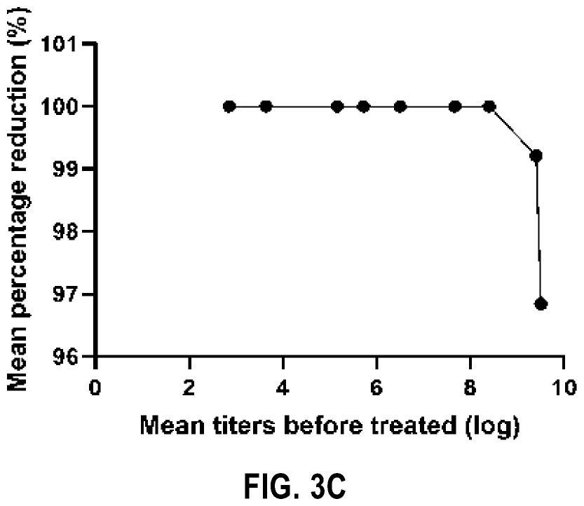

The results were shown in FIGS. 3A-C. FIG. 3A showed the changes in the measured residual pathogen titer as the initial pathogen titer increased. FIG. 3B and FIG. 3C were evaluation results of MLRF method and MPR method, respectively. It was seen that a pathogen inactivation effect close to 100% could be achieved by using a lower initial concentration of pathogens, while the log RF of the MLRF method was relatively low due to limited detection. The percentage reduction of counts by MRP method with high concentration of pathogens might be less than 100%, while the log RF and count reduction percentage varied with the initial concentration.

For example, when the initial concentration of E. coli was 7.67±0.17, the log RF after inactivation was 7.67±0.17, and the pathogen count reduction percentage was close to 100%. However, when the initial concentration of E. coli was 9.50±0.11, the pathogen count reduction percentage was 96.84±0.37%, and the log RF was only 1.50±0.16. Therefore, for different initial E. coli titers, different results of MLRF and MPR were obtained after treatment with riboflavin and UV light under the same conditions. Accordingly, the maximum inactivation capacity of this method could not be accurately evaluated by the pathogen count reduction percentage and log RF.

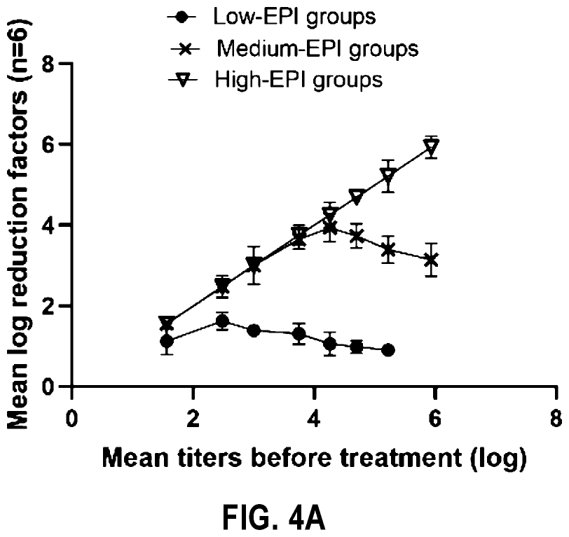

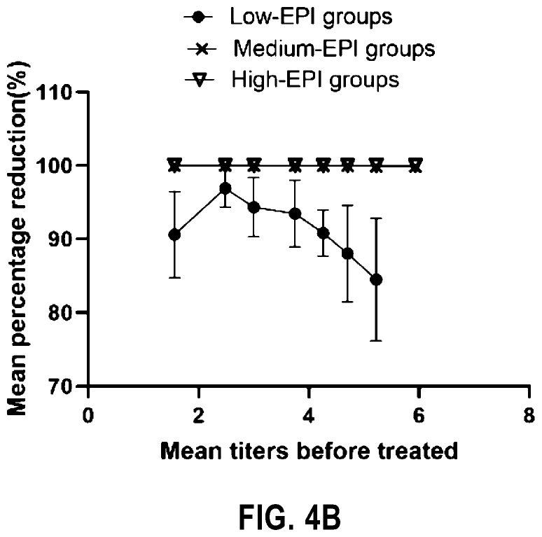

Similarly, VSV was inactivated with reference to the method in Example 2, and the results were evaluated by MLRF method and MPR method, as shown in FIGS. 4A-B. The sensitivity of MPR was poor because there was almost no difference between the high-EPI group and the middle-EPI group (FIG. 4B); the difference in EPI could be sensitively shown by log RF, but the results varied widely depending on the initial titer (FIG. 4A).

In summary, the present disclosure provides an evaluation method of a pathogen inactivation effect using MVEPI as a parameter. The present disclosure is a method for directly evaluating the pathogen inactivation effect. The method is suitable for evaluating the maximum inactivation capacity of different technologies and methods, or for comparing inactivation capabilities of different inactivation technologies and methods. The method shows wide application range, desirable stability, and accurate and effective evaluation results, and has the value of popularization and application.

What is claimed is:

1. An evaluation method of a pathogen inactivation effect, comprising: evaluating an inactivation effect with a maximum value of effective pathogen inactivation (MVEPI) as a standard reference index; wherein
the MVEPI refers to a maximum of a log reduction factor after pathogen solutions of different concentrations before inactivation are treated according to a same inactivation method.

2. The evaluation method according to claim 1, wherein the log reduction factor is expressed as follows:
log RF=lg(N/N₀)=lgN−lgN₀; and
log RF represents the log reduction factor, N represents a pathogen concentration before inactivation, and N₀ represents a pathogen concentration after inactivation.

3. The evaluation method according to claim 2, wherein the pathogen concentration before inactivation and the pathogen concentration after inactivation each are detected by counting for in vitro cell culture, or calculated by a PCR method or a chemical method, or obtained by thermal curve analysis of a terahertz (THz) metamaterial and other pathogen detection methods.

4. The evaluation method according to claim 1, wherein the pathogen is selected from a group consisting of a bacterium, a virus, a parasite, a protozoon, and a rickettsia.

5. The evaluation method according to claim 4, wherein the bacterium is selected from a group consisting of Escherichia coli and Staphylococcus aureus, and the virus is vesicular stomatitis virus (VSV).

6. The evaluation method according to claim 1, wherein the pathogen is marked with a label selected from a group consisting of a radioactive label, a fluorescent label, and a chemical label.

7. The evaluation method according to claim 1, wherein the MVEPI is obtained by conducting linear fitting analysis with a residual pathogen concentration after inactivation as an independent variable and a corresponding pathogen concentration before inactivation as a dependent variable; alternatively,
conducting linear fitting analysis with a pathogen solution concentration before inactivation as an independent variable and a corresponding log reduction factor as a dependent variable; alternatively,
conducting linear fitting analysis with the pathogen solution concentration before inactivation as an independent variable and a corresponding pathogen solution concentration after inactivation as a dependent variable.

8. The evaluation method according to claim 7, wherein the MVEPI is obtained by a method comprising the following steps:
(1) preparing pathogen solutions of different concentrations before inactivation;
(2) conducting pathogen inactivation according to a same inactivation method on each of the pathogen solutions prepared in step (1), and conducting detection to obtain a pathogen concentration in each of the pathogen solutions after inactivation;
(3) calculating a log reduction factor for each of the pathogen solutions:
log RF=lgN−lgN₀; wherein
log RF represents the log reduction factor, N represents a pathogen concentration before inactivation of the pathogen solution, and No represents a pathogen concentration after inactivation of the pathogen solution;
(4) analyzing a relationship between the lgN and the corresponding log RF thereof, taking a part having a linear function relationship between the lgN and the log RF, and conducting linear fitting with the lgN as an independent variable and the log RF as a dependent variable on the part to obtain a linear fitting function:
D=f(C); wherein
D represents the log RF, and C represents the lgN; and
(5) allowing D=C in the linear fitting function, and conducting calculation to obtain a value of the C, namely the MVEPI.

9. The evaluation method according to claim 8, wherein the MVEPI is obtained by a method comprising the following steps:
(1′) preparing pathogen solutions of different concentrations before inactivation;
(2′) conducting pathogen inactivation according to a same inactivation method on each of the pathogen solutions prepared in step (1′), and conducting detection to obtain a pathogen concentration in each of the pathogen solutions after inactivation;
(3′) calculating each of the pathogen solutions of different concentrations before inactivation, and corresponding pathogen concentration logarithm values lgN and lgN₀ in each of the pathogen solutions after inactivation, wherein N represents the pathogen concentration before inactivation of the pathogen solution, and No represents the corresponding pathogen concentration after inactivation of the pathogen solution;
(4′) analyzing a relationship between the lgN and the lgN₀, taking a part having a linear function relationship between the lgN and the lgN₀, and conducting linear fitting with the lgN as an independent variable and the lgN₀ as a dependent variable on the part to obtain a linear fitting function:
D′=f(C′); wherein
D′ represents the lgN₀, and C′ represents the lgN₁; and
(5′) allowing D′=0 in the linear fitting function, and conducting calculation to obtain a value of the C′, namely the MVEPI.

10. The evaluation method according to claim 8, further comprising conducting error control as follows:
(a′) preparing m parts of the pathogen solution before inactivation that are identical to step (1) as a control group solution according to the method of step (1); and
(b′) subjecting the control group solution obtained in step (a′) and the pathogen solution after inactivation obtained in step (2) to pathogen culture under same conditions for a same time, and comparing a growth rate of pathogens in the control group solution obtained in step (a′) with that of the pathogen solution after inactivation obtained in step (2).

11. The evaluation method according to claim 9, further comprising conducting error control as follows:
(a′) preparing m parts of the pathogen solution before inactivation that are identical to step (1) as a control group solution according to the method of step (1); and
(b′) subjecting the control group solution obtained in step (a′) and the pathogen solution after inactivation obtained in step (2) to pathogen culture under same conditions for a same time, and comparing a growth rate of pathogens in the control group solution obtained in step (a′) with that of the pathogen solution after inactivation obtained in step (2).
